(19) United States
(12) Patent Application Publication
(10) Pub. No.: US 2004/0259909 A1
(43) Pub. Date: Dec. 23, 2004

(54) Treatment of fibromyalgia syndrome

(76) Inventors: Dennis McCarthy, Wilmington, DE (US); David Gurley, Wilmington, DE (US)

Correspondence Address: ASTRA ZENECA PHARMACEUTICALS LP, GLOBAL INTELLECTUAL PROPERTY, 1800 CONCORD PIKE, WILMINGTON, DE, 19850-5437, US

(21) Appl. No.: 10/492,891

(22) PCT Filed: Oct. 15, 2002
(86) PCT No.: PCT/SE02/01887

(30) Foreign Application Priority Data
Oct. 16, 2001 (SE) 01034636
Apr. 4, 2002 (SE) 02010338

(51) Int. Cl. A61K 31/4745; A61K 31/424

(52) U.S. Cl. 514/305; 514/375

(57) ABSTRACT

A method for treating fibromyalgia syndrome with an agonist of α7 nicotinic acetylcholine receptors.

### BACKGROUND

Fibromyalgia syndrome (FMS) is a complex chronic condition that causes widespread muscular pain and profound fatigue. Other symptoms include impaired memory, depression, impaired concentration, irritable bladder, sleep disturbance, and headaches. This debilitating, chronic affliction affects 10 million Americans and there is no known cure for the disease. Many of the current treatments have only a partial or temporary effects on some of the symptoms.

Tropisetron is an antagonist at the 5HT₃ receptor that was developed as a treatment for emesis. In animal models, tropisetron, but not ondansetron, was shown to antagonize spatial navigation impairment in a complex spatial memory task (Pharm. Biochem. Behavior. 56:571, 1997). The authors suggested, “the possible existence of other 5-HT₃ receptor subtypes might help to explain the different behavioral effects of ondansetron, tropisetron and itasetron.” Recently, it has been reported that fibromyalgia patients treated with tropisetron showed a statistically significant reduction in their symptoms (Scand. J. Rheumatol. Suppl. 113:46-55, 2000). The positive effects of this drug in fibromyalgia patients were attributed to tropisetron's binding to the 5HT₃ receptor.

### DESCRIPTION OF THE INVENTION

We have now discovered that tropisetron acts as a potent partial agonist of the α7 nicotinic acetylcholine receptor. This discovery links the symptoms of FMS to activity of α7 receptors rather than those of 5HT₃ receptors.

The α7 nicotinic acetylcholine receptors are abundant in cholinergic brain areas important to cognition and memory. This receptor has also been associated with the modulation of neurotransmission and the modulation of long-term potentiation (LTP). This receptor may also function as a filter to gate external sensory inputs, thus making it an attractive target for treatment of cognitive deficits such as those observed in FMS patients. Many of the symptoms such as pain, memory loss, compromised attention, and irritable bladder exhibited by patients with FMS can be linked to activation or desensitization of the α7 receptor. We believe the etiology of FMS is linked to the α7 receptor and that patients with FMS would respond to treatment with α7 agonists, such as the compounds disclosed herein.

A variety of α7 agonists are known that are useful in all aspects of the present invention.

Accordingly, the present invention relates to the use of agonists of α7 nicotinic acetylcholine receptors to treat FMS. Therefore, in one aspect the present invention is directed to the treatment of FMS with α7 agonists. In a second aspect the invention is directed to the use of an α7 agonist to treat the symptoms of FMS. In another aspect the invention is directed to pharmaceutical compositions containing α7 agonists useful for the treatment or amelioration of FMS.

The invention relates to the use of an α7 agonist for the treatment or prophylaxis of fibromyalgia syndrome and fibromyalgia-related symptoms. The invention can be put into practice by clinical trials in which the alleviation of the symptoms in patients with FMS is measured in drug-treated and placebo controls.

In one aspect of the invention, the α7 agonist is a compound that has a Kᵢ value of less than 1000 nM in the ¹²⁵I-α-Bungarotoxin binding to rat hippocampal membrane assay.

In another aspect of the invention, the α7 agonist is a compound that has an EC₅₀ value of less than 30 μM in the functional rat oocyte assay.

In another aspect of the invention, the α7 agonist is a compound that has a Kᵢ value of less than 1000 nM in the ¹²⁵I-α-Bungarotoxin binding to rat hippocampal membrane assay and an EC₅₀ value in the functional rat oocyte assay of less than 30 μM.

Another aspect of the invention relates to a method for the manufacture of a medicament for the treatment or prophylaxis of fibromyalgia syndrome and fibromyalgia-related symptoms comprising an α7 agonist, wherein the α7 agonist is defined as described by any of the above embodiments.

We have discovered that the 5HT₃ receptor antagonist tropisetron is a potent and selective partial agonist at the α7 receptor. In contrast, the structurally similar 5HT₃ antagonist, ondansetron, was shown to lack activity at the α7 receptor.

Therefore, the memory effects of tropisetron are likely to arise from its action at the α7 receptor. Accordingly, we believe that the positive therapeutic activity of tropisetron in FMS patients is due to the action of this drug at the α7 receptor and not due to actions at the 5HT₃ receptor as previously reported.

### BRIEF DESCRIPTION OF THE DRAWINGS

FIG. 1 shows the currents elicited in frog oocytes expressing mouse nAChR α7-receptors by acetylcholine or tropisetron.

### DETAILED DESCRIPTION OF THE INVENTION

In a first embodiment of the invention a suitable α7 agonist is spiro[1-azabicyclo[2.2.2]octane-3,5′-oxazolidine-2′-one (Compound 1, Table 1). This compound is a selective α7 agonist with a wide safety margin. This compound is disclosed in U.S. Pat. No. 5,902,814 the disclosure of which is incorporated herein in its entirety by reference. This compound is active in animal models of memory and cognition.

In a second aspect of the invention a suitable α7 agonist is a compound as disclosed in PCT publication WO 01/60821 the disclosure of which is incorporated herein in its entirety by reference, having the structure:

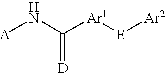

wherein:

A is selected from

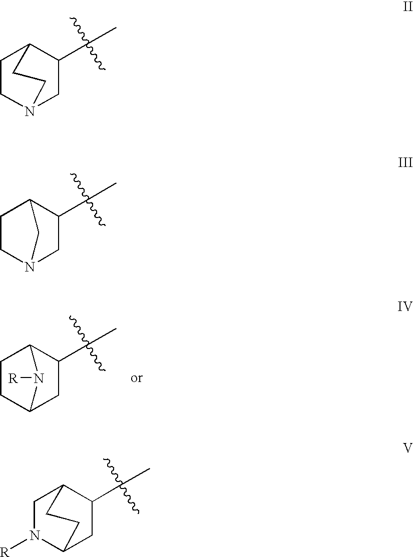

D is oxygen or sulfur;

E is a single bond, oxygen, sulfur, or NR¹⁰;

R is hydrogen or methyl;

Ar¹ is a 5- or 6-membered aromatic or heteroaromatic ring containing 0, 1, 2 or 3 nitrogen, oxygen or sulfur atoms, wherein there is no more than 1 oxygen or sulfur atom;

Ar² is a 5- or 6-membered aromatic or heteroaromatic ring containing 0, 1, 2 or 3 nitrogen, oxygen or sulfur atoms, wherein there is no more than 2 oxygen or sulfur atom; or an 8-, 9- or 10-membered fused aromatic or heteroaromatic ring system containing 0, 1, 2 or 3 nitrogen, oxygen or sulfur atoms, wherein there is no more than 2 oxygen or sulfur atoms;

wherein if Ar² is unsubstituted phenyl, then Ar¹ is not pyrazolyl;

wherein the aromatic rings Ar¹ and Ar² are substituted with 0, 1, 2 or 3 substituents selected from halogen, C₁₋₄alkyl, C₂₋₄alkenyl, C₂₋₄alkynyl, CN, NO₂, NR¹R², CH₂NR¹R², OR³, CH₂OR³, CO₂R⁴ and CF₃; but

if Ar¹ is phenyl and Ar² is quinolynyl, then Ar² is substituted with 0, 1, 2 or 3 substituents selected from C₁₋₄alkyl, C₂₋₄alkenyl, C₂₋₄alkynyl, CN, NO₂, NR¹R², CH₂NR¹R², OR³, CH₂OR³ and CO₂R⁴;

R¹, R², and R³ are independently C₁₋₄alkyl, aryl, heteroaryl, C(O)R⁵, C(O)NHR⁶, C(O)R⁷, SO₂R⁸; or R¹ and R² may together be (CH₂)ⱼG(CH₂)ₖ where G is oxygen, sulfur, NR⁹, or a single bond;

j is 2, 3 or 4;

k is 0, 1 or 2;

R⁴, R⁵, R⁶, R⁷, R⁸, R⁹, and R¹⁰ are independently C₁₋₄alkyl, aryl, or heteroaryl; or an enantiomer thereof and pharmaceutically acceptable salts thereof; with the provisos that:

(1) if D represents oxygen, E represents a single bond, and A represents:

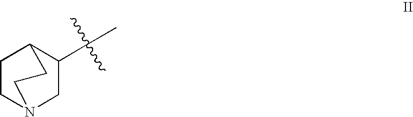

and either Ar¹ or Ar² represents a pyrazole ring, then all optional substituents on the pyrazole ring shall be hydrogen; and

(2) if Ar¹ represents a pyridine ring, Ar² represents an aryl ring, and A represents:

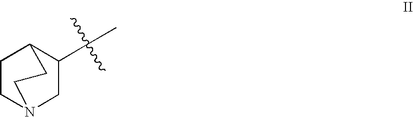

then all optional substituents on the pyridine ring shall be hydrogen.

Particular compounds that are embodiments of this aspect of the inventions are compounds below:

N-(1-azabicyclo[2.2.2]oct-3-yl)(3-phenylbenzamide);

N-(1-azabicyclo[2.2.2]oct-3-yl)(3-(2-thienyl)benzamide);

N-(1-azabicyclo[2.2.2]oct-3-yl)(3-(3-thienyl)benzamide);

N-(1-azabicyclo[2.2.2]oct-3-yl)(4-phenylthiophene-2-carboxamide), compound 3, Table 1;

N-(1-azabicyclo[2.2.2]oct-3-yl)(5-phenylthiophene-3-carboxamide);

N-(1-azabicyclo[2.2.2]oct-3-yl)(5-phenylthiophene-2-carboxamide);

N-(1-azabicyclo[2.2.2]oct-3-yl)(5-phenylfuran-2-carboxamide);

N-(1-azabicyclo[2.2.2]oct-3-yl)(5-(2-pyridyl)furan-2-carboxamide);

N-(1-azabicyclo[2.2.2]oct-3-yl)(5-(3-pyridyl)furan-2-carboxamide);

N-(1-azabicyclo[2.2.2]oct-3-yl)(5-(2-furyl)furan-2-carboxamide);

N-(1-azabicyclo[2.2.2]oct-3-yl)(5-(3-furyl)furan-2-carboxamide);

N-(1-azabicyclo[2.2.2]oct-3-yl)(5-(2-thienyl)furan-2-carboxamide);

N-(1-azabicyclo[2.2.2]oct-3-yl)(5-(3-thienyl)furan-2-carboxamide);

N-(1-azabicyclo[2.2.2]oct-3-yl)(5-(3-fluorophenyl)furan-2-carboxamide);

N-(1-azabicyclo[2.2.2]oct-3-yl)(3-(3-pyridyl)benzamide);

N-(1-azabicyclo[2.2.2]oct-3-yl)(3-(3-methoxyphenyl)benzamide);

N-(1-azabicyclo[2.2.2]oct-3-yl)(3-(2-methoxy phenyl)benzamide);

N-(1-azabicyclo[2.2.2]oct-3-yl)(3-(3-(N-acetylamino)phenyl)benzamide);

N-(1-azabicyclo[2.2.2]oct-3-yl)(3-(3-fluorophenyl)benzamide);

N-(1-azabicyclo[2.2.2]oct-3-yl)(3-(3-methylphenyl)benzamide);

N-(1-azabicyclo[2.2.2]oct-3-yl)(3-(3,5-dichlorophenyl)benzamide);

N-(1-azabicyclo[2.2.2]oct-3-yl)(3-(2-naphthyl)benzamide);

N-(1-azabicyclo[2.2.2]oct-3-yl)(3-(4-fluorophenyl)benzamide);

N-(1-azabicyclo[2.2.2]oct-3-yl)(5-(2-benzo[b]furanyl)furan-2-carboxamide);

N-(1-azabicyclo[2.2.2]oct-3-yl)(5-(4-pyridyl)furan-2-carboxamide);

N-(1-azabicyclo[2.2.2]oct-3-yl)(5-(3-methoxyphenyl)furan-2-carboxamide);

N-(1-azabicyclo[2.2.2]oct-3-yl)(5-(2-methoxyphenyl)fuiran-2-carboxamide);

N-(1-azabicyclo[2.2.2]oct-3-yl)(5-(4-fluorophenyl)furan-2-carboxamide);

N-(1-azabicyclo[2.2.2]oct-3-yl)(5-(2-naphthyl)furan-2-carboxamide);

N-(1-azabicyclo[2.2.2]oct-3-yl)(5-(3-methylphenyl)furan-2-carboxamide);

N-(1-azabicyclo[2.2.2]oct-3-yl)(5-(4-pyridyl)thiophene-2-carboxamide);

N-(1-azabicyclo[2.2.2]oct-3-yl)(5-(3-pyridyl)thiophene-2-carboxamide);

N-(1-azabicyclo[2.2.2]oct-3-yl)(5-(2-pyridyl)thiophene-2-carboxamide);

N-(1-azabicyclo[2.2.2]oct-3-yl)(4-(2-pyridyl)thiophene-2-carboxamide);

N-(1-azabicyclo[2.2.2]oct-3-yl)(4-(4-pyridyl)thiophene-2-carboxamide);

N-(1-azabicyclo[2.2.2]oct-3-yl)(4-(3-pyridyl)thiophene-2-carboxamide);

N-(1-azabicyclo[2.2.2]oct-3-yl)(5-(3-(N-acetylamino)phenyl)furan-2-carboxamide);

N-(1-azabicyclo[2.2.2]oct-3-yl)(5-(3-nitrophenyl)furan-2-carboxamide);

N-(1-azabicyclo[2.2.2]oct-3-yl)(5-(3-trifluoromethylphenyl)furan-2-carboxamide);

N-(1-azabicyclo[2.2.2]oct-3-yl)(5-(3-chlorophenyl)furan-2-carboxamide);

N-(1-azabicyclo[2.2.2]oct-3-yl)(5-(3-(N-acetylamino)phenyl)thiophene-2-carboxamide);

N-(1-azabicyclo[2.2.2]oct-3-yl)(5-(3-fluorophenyl)thiophene-2-carboxamide);

N-(1-azabicyclo[2.2.2]oct-3-yl)(5-(3-methoxyphenyl)thiophene-2-carboxamide);

N-(1-azabicyclo[2.2.2]oct-3-yl)(5-(3-ethoxyphenyl)thiophene-2-carboxamide);

N-(1-azabicyclo[2.2.2]oct-3-yl)(5-(3,5-dimethylisoxazol-4-yl)furan-2-carboxamide);

N-(1-azabicyclo[2.2.2]oct-3-yl)(5-(3,5-dimethylisoxazol-4-yl)thiophene-2-carboxamide);

N-(1-azabicyclo[2.2.2]oct-3-yl)(5-(3-aminophenyl)thiophene-2-carboxamide);

N-(1-azabicyclo[2.2.2]oct-3-yl)(5-(3-pyridyl)thiophene-3-carboxamide);

N-(1-azabicyclo[2.2.2]oct-3-yl)(5-(4-chlorophenyl)furan-2-carboxamide);.

N-(1-azabicyclo[2.2.2]oct-3-yl)(5-(3-pyridyl)thiazole-3-carboxamide);

N-(1-azabicyclo[2.2.2]oct-3-yl)(5-(4-pyridyl)thiazole-3-carboxamide);

N-(1-azabicyclo[2.2.2]oct-3-yl)(5-(3-(N,N-dimethylamino)phenyl)thiophene-2-carboxamide);

N-(1-azabicyclo[2.2.2]oct-3-yl)(5-(8-quinolinyl)thiophene-2-carboxamide);

N-(1-azabicyclo[2.2.2]oct-3-yl)(5-(3 -cyanophenyl)thiophene-2-carboxamide);

N-(1-azabicyclo[2.2.2]oct-3-yl)(5-(3-(N-methylamino)phenyl)thiophene-2-carboxamide);

N-(1-azabicyclo[2.2.2]oct-3-yl)(5-(3-hydroxyphenyl)thiophene-2-carboxamide);

N-(1-azabicyclo[2.2.2]oct-3-yl)(5-(3-pyridylamino)thiophene-2-carboxamide);

N-(1-azabicyclo[2.2.2]oct-3-yl)(5-(3-chlorophenyl)thiophene-2-carboxamide);

N-(1-azabicyclo[2.2.2]oct-3-yl)(5-(3-(4-morpholinyl)phenyl)thiophene-2-carboxamide)

N-(1-azabicyclo[2.2.2]oct-3-yl)(5-(3-(aminomethyl)phenyl)thiophene-2-carboxamide);

N-(1-azabicyclo[2.2.2]oct-3-yl)(5-phenoxythiophene-2-carboxamide);

N-(1-azabicyclo[2.2.2]oct-3-yl)(5-(3-aminophenyl)furan-2-carboxamide);

N-(1-azabicyclo[2.2.2]oct-3-yl)(5-(3-(N,N-dimethylamino)phenyl)furan-2-carboxamide);

N-(1-azabicyclo[2.2.2]oct-3-yl)(5-(3-formylphenyl)thiophene-2-carboxamide), or

N-(1-azabicyclo[2.2.2]oct-3-yl)(5-(3-(hydroxymethyl)phenyl)thiophene-2-carboxamide)

or an enantiomer thereof, or a pharmaceutically-acceptable salt thereof.

In a third aspect of the invention a suitable α7 agonist is a compound as disclosed in PCT publication WO 01/29034 the disclosure of which is incorporated herein by reference in its entirety, having the structure:

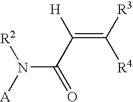

wherein:

A represents a moiety selected from:

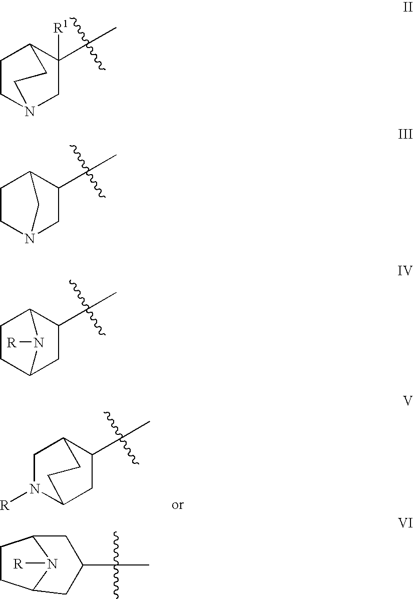

R represents hydrogen or methyl;

R¹ and R² are independently hydrogen, or C₁-C₄ alkyl;

R³ and R⁴ are independently hydrogen, C₁-C₄ alkyl or SAr, provided that at least one of R³ and R⁴ is SAr;

Ar represents a 5- or 6-membered aromatic or heteroaromatic ring containing zero to three nitrogen atoms, zero or one oxygen atom, and zero or one sulfur atom or an 8-, 9- or 10-membered fused aromatic or heteroaromatic ring system containing zero to four nitrogen atoms, zero to one oxygen atom, and zero to one sulfur atom which may optionally be substituted with one or more substituents selected from: hydrogen, halogen, C₁-C₄ alkyl, C₂-C₄ alkenyl, C₂-C₄ alkynyl, aryl, heteroaryl, —CO₂R⁵, —CN, —NO₂, —NR⁶R⁷, —CF₃, —OR⁸;

R⁵, R⁶, R⁷, and R⁸ are independently hydrogen, C₁-C₄ alkyl, aryl, heteroaryl, —C(O)R⁹, —C(O)NHR¹⁰, —C(O)R¹¹, —SO₂R¹²; or,

R⁶ and R⁷ may together be (CH₂)ⱼQ(CH₂)ₖ where Q is O, S, NR¹³, or, a bond;

j is 2to 7;

k is 0to 2;

R⁹, R¹⁰, R¹¹, R¹², and R¹³, are independently C₁-C₄ alkyl, aryl, or heteroaryl; or an enantiomer thereof, and the pharmaceutically acceptable salts thereof.

Particular compounds that are embodiments of this aspect of the inventions are:

N-(1-aza-bicyclo[2.2.2]oct-3-yl)[Z-3-(phenylthio)propenamide]hydrochloride;

N-(1-aza-bicyclo[2.2.2]oct-3-yl)[Z-3-(4-methylphenylthio)propenamide];

N-(1-aza-bicyclo[2.2.2]oct-3-yl)[E-3-(4-methylphenylthio)propenamide];

N-(1-aza-bicyclo[2.2.2]oct-3-yl)[Z-3-(3-methylphenylthio)propenamide];

N-(1-aza-bicyclo[2.2.2]oct-3-yl)[E-3-(3-methylphenylthio)propenamide];

N-(1-aza-bicyclo[2.2.2]oct-3-yl)[Z-3-(2-methylphenylthio)propenamide];

N-(1-aza-bicyclo[2.2.2]oct-3-yl)[E-3-(2-methylphenylthio)propenamide];

N-(1-aza-bicyclo[2.2.2]oct-3-yl)[Z-3-(4-methoxyphenylthio)propenamide];

N-(1-aza-bicyclo[2.2.2]oct-3-yl)[E-3-(4-methoxyphenylthio)propenamide];

N-(1-aza-bicyclo[2.2.2]oct-3-yl)[Z-3-(3-methoxyphenylthio)propenamide];

N-(1-aza-bicyclo[2.2.2]oct-3-yl)[E-3-(3-methoxyphenylthio)propenamide];

N-(1-aza-bicyclo[2.2.2]oct-3-yl)[Z-3-(2-methoxyphenylthio)propenamide];

N-(1-aza-bicyclo[2.2.2]oct-3-yl)[E-3-(2-methoxyphenylthio)propenamide];

N-(1-aza-bicyclo[2.2.2]oct-3-yl)[Z-3-(2-pyridylthio)propenamide];

N-(1-aza-bicyclo[2.2.2]oct-3-yl)[E-3-(2-pyridylthio)propenamide];

N-(1-aza-bicyclo[2.2.2]oct-3-yl)[Z-3-(4-pyridylthio)propenamide];

N-(1-aza-bicyclo[2.2.2]oct-3-yl)[E-3-(4-pyridylthio)propenamide];

N-(1-aza-bicyclo[2.2.2]oct-3-yl)[Z-3-(2-pyrimidinylthio)propenamide];

N-(1-aza-bicyclo[2.2.2]oct-3-yl)[E-3-(2-pyrimidinylthio)propenamide];

N-(1-aza-bicyclo[2.2.2]oct-3-yl)[Z-3-(2-methyl-3-furanylthio)propenamide];

N-(1-aza-bicyclo[2.2.2]oct-3-yl)[E-3-(2-methyl-3-furanylthio)propenamide];

N-(1-aza-bicyclo[2.2.2]oct-3-yl)[E-3-(2-imidazolylthio)propenamide];

N-(1-aza-bicyclo[2.2.2]oct-3-yl)[Z-3-(phenylthio)-3-(methyl)propenamide];

N-(1-aza-bicyclo[2.2.2]oct-3-yl)[Z-3-(2-benzothiazolylthio)propenamide];

N-(1-aza-bicyclo[2.2.2]oct-3-yl)[E-3-(2-benzothiazolylthio)propenamide];

N-(1-aza-bicyclo[2.2.2]oct-3-yl)[Z-3-(1-methyl-2-imidazolylthio)propenamide];

N-(1-aza-bicyclo[2.2.2]oct-3-yl)[E-3-(1-methyl-2-imidazolylthio)propenamide];

N-(1-aza-bicyclo[2.2.2]oct-3-yl)[Z-3-(5-methyl-1,3,4-thiadiazol-2-ylthio)propenamide];

N-(1-aza-bicyclo[2.2.2]oct-3-yl)[E-3-(5-methyl-1,3,4-thiadiazol-2-ylthio)propenamide];

N-(1-aza-bicyclo[2.2.2]oct-3-yl)[Z-3-(4-chlorophenylthio)propenamide];

N-(1-aza-bicyclo[2.2.2]oct-3-yl)[Z-3-(2-thiazolylthio)propenamide];

N-(1-aza-bicyclo[2.2.2]oct-3-yl)[Z-3-(2-thienylthio)propenamide];

N-(1-aza-bicyclo[2.2.2]oct-3-yl)[E-3-(2-thienylthio)propenamide];

N-(1-aza-bicyclo[2.2.2]oct-3-yl)[Z-3-(2-benzoxazolylthio)propenamide];

N-(1-aza-bicyclo[2.2.2]oct-3-yl)[E-3-(2-benzoxazolylthio)propenamide];

N-(1-aza-bicyclo[2.2.2]oct-3-yl)[Z-3-(4-trifluoromethyl-2-pyrimidinylthio)propenamide];

N-(1-aza-bicyclo[2.2.2]oct-3-yl)[Z-3-(4-fluorophenylthio)propenamide];

N-(1-aza-bicyclo[2.2.2]oct-3-yl)[E-3-(4-fluorophenylthio)propenamide];

N-(1-aza-bicyclo[2.2.2]oct-3-yl)[Z-3-(2-thiazolo[4,5-b]pyridylthio)propenamide];

(R)—N-(1-aza-bicyclo[2.2.2]oct-3-yl)[E-3-(2-thiazolo[4,5-b]pyridylthio)propenamide];

N-(1-aza-bicyclo[2.2.2]oct-3-yl)[Z-3-(3-fluorophenylthio)propenamide], or

N-(1-aza-bicyclo[2.2.2]oct-3-yl)[E-3-(3-fluorophenylthio)propenamide];

or an enantiomer thereof, or a pharmaceutically-acceptable salt thereof

In a fourth aspect of the invention a suitable α7 agonist is a compound as disclosed in U.S. Pat. No. 6,110,914 the disclosure of which is incorporated herein by reference in its entirety, having the structure:

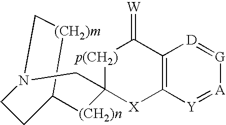

wherein n is 0 or 1;

m is 0 or 1;

p is 0 or 1;

X is oxygen or sulfur;

Y is CH, N or NO;

W is oxygen, H₂ or F₂;

A is N or C(R²);

G is N or C(R³);

D is N or C(R⁴);

with the proviso that no more than one of A, G, and D is nitrogen but at least one of Y, A, G, and D is nitrogen or NO;

R¹ is hydrogen or C₁₋₄alkyl;

R², R³, and R⁴ are independently hydrogen, halogen, C₁₋₄alkyl, C₂₋₄alkenyl, C₂₋₄alkynyl, aryl, heteroaryl, OH, OC₁₋₄alkyl, CO₂R¹, —CN, —NO₂, —NR⁵R⁶, —CF₃, —OSO₂CF₃, or R² and R³, or R³ and R⁴, respectively, may together form another six membered aromatic or heteroaromatic ring sharing A and G, or G and D, respectively, containing 0, 1 or 2 nitrogen atoms, and substituted with one to two substituents independently selected from hydrogen, halogen, C₁₋₄alkyl, C₂₋₄alkenyl, C₂₋₄alkynyl, aryl, heteroaryl, OH, OC₁₋₄alkyl, CO₂R¹, —CN, —NO₂, —NR⁵R⁶, —CF₃, —OSO₂CF₃;

R⁵ and R⁶ are independently hydrogen, C₁₋₄alkyl, C(O)R⁷, C(O)NHR⁸, C(O)OR⁹, SO₂R¹⁰ or may together be (CH₂)ⱼQ(CH₂)ₖ where Q is O, S, NR¹¹, or a bond;

j is 2 to 7;

k is 0 to 2;

R⁷, R⁸, R⁹, R¹⁰, and R¹¹ are independently C₁₋₄ alkyl, aryl, or heteroaryl, or an enantiomer thereof, and the pharmaceutically acceptable salts thereof.

Particular compounds that are embodiments of this aspect of the inventions are:

spiro[1-azabicyclo[2.2.2]octane-3,2′-(3′H)-furo[2,3-b]pyridine], Compound 2, Table 1;

5′-bromospiro[1-azabicyclo[2.2.2]octane-3,2′-(3′H)-furo[2,3-b]pyridine];

5′-phenylspiro[1-azabicyclo[2.2.2]octane-3,2′-(3′H)-furo[2,3-b]pyridine];

5′-nitrospiro[1-azabicyclo[2.2.2]octane-3,2′-(3′H)- furo[2,3-b]pyridine];

1′-chlorospiro[1-azabicyclo[2.2.2]octane-3,2′-(3′H)-furo[2,3-b]isoquinoline];

5′-(phenylcarboxamido)spiro[1-azabicyclo[2.2.2]octane-3,2′-(3 ′H)-furo[2, 3-b]pyridine];

5′-(phenylaminocarbonylamino)spiro[1-azabicyclo[2.2.2]octane-3,2′-(3′H)-furo[2, 3-b]pyridine];

5′-(phenylsulfonylamido)spiro[1-azabicyclo[2.2.2]octane-3,2′-(3′H)-furo[2, 3-b]pyridine];

5′-aminospiro[1-azabicyclo[2.2.2]octane-3,2′-(3′H)-furo[2,3-b]pyridine];

5′-N-methylaminospiro[1-azabicyclo[2.2.2]octane-3,2′-(3′H)-furo[2,3-b]pyridine];

5′-N,N-dimethylaminospiro[1-azabicyclo[2.2.2]octane-3,2′-(3′H)-furo[2, 3-b]pyridine];

5′-N,N-diethylaminospiro[1-azabicyclo[2.2.2]octane-3,2′-(3′H)-furo[2, 3-b]pyridine];

5′-N-ethylaminospiro[1-azabicyclo[2.2.2]octane-3,2′-(3′H)-furo[2,3-b]pyridine];

5′-N-benzylaminospiro[1-azabicyclo[2.2.2]octane-3,2′-(3′H)-furo[2,3-b]pyridine];

5′-N-formamidospiro[1-azabicyclo[2.2.2]octane-3,2′-(3′H)-furo[2,3-b]pyridine];

5′-N-acetamidospiro[1-azabicyclo[2.2.2]octane-3,2′-(3′H)-furo[2,3-b]pyridine];

spiro[1-azabicyclo[2.2.2]octane-3,2′-(3′H)-furo[2,3-b]isoquinoline];

spiro[1-azabicyclo[2.2.2]octane-3,2′-(3′H)-furo[2,3-b]quinoline];

5′-ethenylspiro[1-azabicyclo[2.2.2]octane-3,2′-(3′H)-furo[2,3-b]pyridine];

5′-(E)-(phenylethenyl)spiro[1-azabicyclo[2.2.2]octane-3,2′-(3′H)-furo[2, 3-b]pyridine];

5′-(4-morpholino)spiro[1-azabicyclo[2.2.2]octane-3,2′-(3′H)-furo[2, 3-b]pyridine];

5′-(1-azetidinyl)spiro[1-azabicyclo[2.2.2]octane-3,2′-(3′H)-furo[2, 3-b]pyridine];

5′-(E)-(2-(4-pyridyl)ethenyl)spiro[1-azabicyclo[2.2.2]octane-3,2′-(3′H)-furo[2,3-b]pyridine];

5′-(E)-(2-(2-pyridyl)ethenyl)spiro[1-azabicyclo[2.2.2]octane-3,2′-(3′H)-furo[2,3-]pyridine];

5′-(2-trimethylsilylethynyl)spiro[1-azabicyclo[2.2.2]octane-3,2′-(3′H)-furo[2, 3-b]pyridine];

5′-ethynylspiro[1-azabicyclo[2.2.2]octane-3,2′-(3′H)-furo[2,3-b]pyridine];

5′-(2-furyl)spiro[1-azabicyclo[2.2.2]octane-3,2′-(3′H)-furo[2,3-b]pyridine];

5′-(3-pyridyl)spiro[1-azabicyclo[2.2.2]octane-3,2′-(3 ′H)-furo[2,3-b]pyridine];

5′-methylspiro[1-azabicyclo[2.2.2]octane-3,2′-(3 ′H)-furo[2,3-b]pyridine];

spiro[1-azabicyclo[2.2.2]octane-3,2′-(3′H)-furo[2,3-b]pyridine-5′carbonitrile];

spiro[1-azabicyclo[2.2.2]octane-3,2′-(3′H)-furo[2,3-b]pyridine-5′carboxamide];

5′-N′-(3-chlorophenyl)aminocarbonylminospiro[1-azabicyclo[2.2.2]octane-3,2′-(3′H)-furo[2,3-b]pyridine];

5′-N′-(2-nitrophenyl)aminocarbonylaminospiro[1-azabicyclo[2.2.2]octane-3,2′-(3′H)-furo[2,3-b]pyridine];

4′-chlorospiro[1-azabicyclo[2.2.2]octane-3,2′-(3′H)-furo[2,3-b]pyridine];

4′-methoxyspiro[1-azabicyclo[2.2.2]octane-3,2′-(3′H)-furo[2,3-b]pyridine];

4′-phenylthiospiro[1-azabicyclo[2.2.2]octane-3,2′-(3′H)-furo[2,3-b]pyridine]; 4′-(N-2-aminoethyl)aminospiro[1-azabicyclo[2.2.2]octane-3,2′-(3′H)-furo[2,3-b]pyridine];

4′-phenylaminospiro[l-azabicyclo[2.2.2]octane-3,2′-(3′H)-furo[2,3-b]pyridine];

4′-methylaminospiro[l-azabicyclo[2.2.2]octane-3,2′-(3′H)-furo[2,3-b]pyridine];

4′-(4-N-methylpiperazin-1-yl)spiro[1-azabicyclo[2.2.2]octane-3,2′-(3′H)-furo[2,3b]pyridine];

4′-chloro-spiro[1-azabicyclo[2.2.2]octane-3,2′-(3′H)-furo[3,2-c]pyridine];

spiro[1-azabicyclo[2.2.2]octane-3,2′-(3′H)-furo[3,2-c]pyridine];

spiro[1-azabicyclo[2.2.2]octane-3,2′(3′H)-furo[2,3-b]pyridine-7′-oxide];

spiro[1-azabicyclo[2.2.2]octane-3,2′(3′H)-furo[2,3-b]pyridine-6′-carbonitrile];

6′-chlorospiro[1-azabicyclo[2.2.2]octane-3,2′(3′H)-furo[2,3-b]pyridine], or

6′-fluorospiro[1-azabicyclo[2.2.2]octane-3,2′(3′H)-furo[2,3-b]pyridine];

or an enantiomer, or a pharmaceutically-acceptable salt thereof.

Experimental:

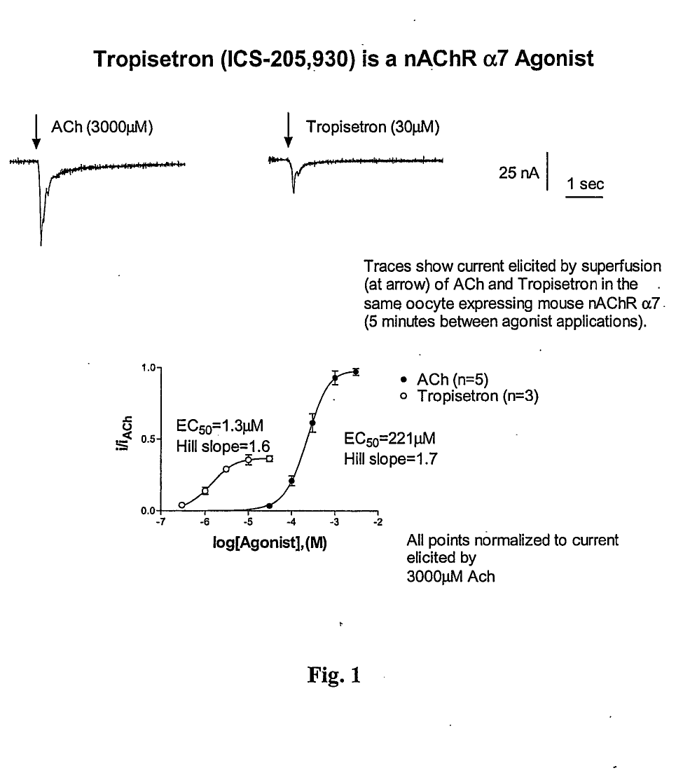

We discovered that the 5HT₃ receptor antagonist tropisetron is a potent and selective partial agonist at the α7 receptor (FIG. 1). In contrast, the structurally similar 5HT₃ antagonist, ondansetron, lacked activity at the α7 receptor (Table 1).

In earlier work (Pharm. Biochem. Behavior. 56:571, 1997) tropisetron, but not ondansetron, antagonized spatial navigation impairment in a complex spatial memory task in animal models suggesting that behavioral differences were not due to actions at he 5HT₃ receptor.

Test A—Assay for Affinity at α7 nAChR Subtype

¹²⁵I-α-Bunzarotoxin (BTX) Binding to Rat Hippocampal Membranes.

Rat hippocampi were homogenized in 20 volumes of cold homogenisation buffer (HB): (in mM): tris(hydroxymethyl)aminomethane 50; MgCl₂ 1; NaCl 120; KCl 5: pH 7.4). The homogenate was centrifuged for 5 min at 1000 g, the supernatant was saved and the pellet re-extracted. The pooled supernatants were centrifuged for 20 min at 12000 g, washed, and re-suspended in HB. Membranes (30-80 μg) were incubated with 5 nM [¹²⁵I]α-BTX, 1 mg/mL BSA (bovine serum albumin), test drug, and either 2 mM CaCl₂ or 0.5 mM EGTA [ethylene glycol-bis(β-aminoethylether)]for 2 h at 21° C., and then filtered and washed four times over Whatman glass fiber filters (thickness C) using a Brandel cell harvester. Pre-treating the filters for 3 h with 1% (BSA/0.01% PEI (polyethyleneimine) in water was critical for low filter blanks (0.07% of total counts per minute). Non-specific binding was described by 100 μM (−)-nicotine, and specific binding was typically 75%.

Test B—Assay for Affinity to the 5-HT₃ nAChR Subtype

[³]zacoipride binding. Binding of 0.5 nM [³H]zacopride was assessed essentially as described in Test A using rat small-bowel muscularis membranes suspended in 50 mM Tris; 150 mM NaCl at pH 7.4. Incubation was continued for one hour.

Binding Data Analysis for Tests A and B

IC₅₀ values and pseudo Hill coefficients (n_{H}) were calculated using the non-linear curve fitting program ALLFIT (DeLean A, Munson P J and Rodbard D (1977) Am. J. Physiol., 235:E97-E102). Saturation curves were fitted to a one site model, using the non-linear regression program ENZFITTER (Leatherbarrow, R. J. (987)), yielding K_{D} values of 1.67 and 0.7 nM for the [¹²⁵I]-α-BTX and [³H]zacopride ligands respectively. Kᵢ values were estimated using the general Cheng-Prusoff equation (A):

Kᵢ=[IC₅₀]/((2+([ligand]/K_{D})ⁿ)^{l/n}−1) (A)

where a value of n=1 was used whenever n_{H}<1.5 and a value of n−2 was used when n_{H}≧1.5. Samples were assayed in triplicate and were typically ±5%. Kᵢ values were determined using six or more drug concentrations. The compounds of the invention are compounds with binding affinities (Kᵢ) of less than 1 μM in Test A, indicating that they are expected to have useful therapeutic activity by interacting at the α7 receptor (Table 1).

TABLE 1
Binding Affinities

| | |
|---|---|
| | Binding Affinity |
| Stereo- | (Ki/nM) |

| Compound | chemistry | α₇ | 5HT₃ |
|---|---|---|---|
| 1 | (R) | 91 | 24000 |
| 2 | (R) | 14 | NA |
| 3 | (R) | 1.6 | 20000 |

Test C—Rat Oocyte Functional Assay

Xenopus oocytes Xenopus laevis frogs (Xenopus I, Kalamazoo, Mich.) were anesthetized using 0.15% tricaine. Oocytes were removed to OR2 solution: (in mM) 82 NaCl, 2.5 KCl, 5 HEPES, 1.5 NaH₂PO₄, 1 MgCl₂, 0.1 EDTA, pH 7.4. The oocytes were defolliculated by incubation in 25 mL OR₂ containing 0.2% collagenase 1A (SIGMA) two times for 60 min on a platform vibrating at 1 Hz and stored in Leibovitz's L-15 medium. Oocytes were injected the following day. Leibovitz's L-15 medium contained 50 μg/mL gentomycin, 10 units/mL penicillin, and 10 μg/mL streptomycin.

Preparation and injection of cRNA Rat nAChR α7 was cloned in-house (Luhowskyj). Non-polyadenylated cRNA was prepared from cDNA using mMessage mMachine SP6 (Ambion) according to the manufacturer's instructions.

Recording The external recording solution consisted of (in mM) 90 NaCl, 1 KCl, 1 MgCl₂, 1 BaCl₂, 5 HEPES, pH 7.4. Two-electrode voltage-clamp recording was carried out using an Oocyte Clamp amplifier (model OC 725C ,Warner Inst., Hamden, Conn.). Oocytes were impaled with two electrodes of 1-2 MΩ tip resistance when filled with 3M KCl. Recordings were begun when membrane potential became stable at potentials negative to −20 mV. Membrane potential was clamped at −80 mV unless otherwise noted. ACh, (−) was purchased from SIGMA.

Calculation of current amplitude and curve fitting Current amplitude was measured from baseline to peak. EC₅₀'s, maximal effect, and Hill slopes were estimated by fitting the data to the logistic equation using GraphPad Prism (GraphPad Software, Inc. San Diego, Calif.)

FIG. 1 shows the effect of acetylcholine and tropisetron on oocytes expressing mouse nAChR α7. In the upper panel, representative traces of current elicited in oocytes expressing mouse nAChR α7 are illustrated. Traces shown are from the same oocyte; superfusion of acetylcholine and tropisetron begins at arrow (5 min between agonist applications). In the lower panel, concentration-response curve to acetylcholine and tropisetron are shown. Data are fit by the logistic equation.

We claim:

1. A method comprising the use of an α7 agonist for the treatment or prophylaxis of fibromyalgia syndrome and fibromyalgia-related symptoms.

2. The method according to claim 1 wherein the α7 agonist is a compound that has a Kᵢ value of less than 1000 nM in the ¹²⁵I-α-Bungarotoxin binding to rat hippocampal membrane assay.

3. The method according to claim 1 wherein said α7 agonist is a compound having the structure

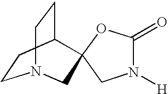

4. The method according to claim 1, wherein the α7 agonist is a compound having the structure: 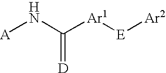
wherein:
A is selected from 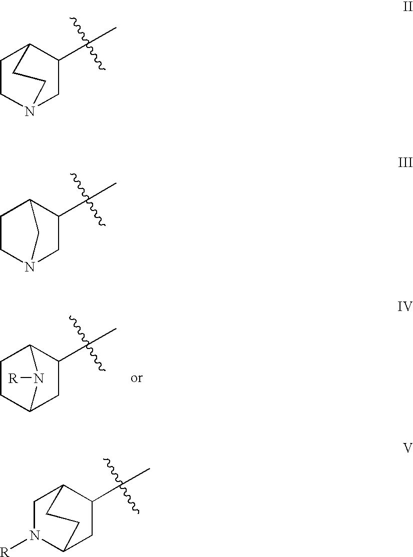
D is oxygen or sulfur;
E is a single bond, oxygen, sulfur, or NR¹⁰;
R is hydrogen or methyl;
Ar¹ is a 5- or 6-membered aromatic or heteroaromatic ring containing 0, 1, 2 or 3 nitrogen, oxygen or sulfur atoms, wherein there is no more than 1oxygen or sulfur atom;
Ar² is a 5- or 6-membered aromatic or heteroaromatic ring containing 0, 1, 2 or 3 nitrogen, oxygen or sulfur atoms, wherein there is no more than 2 oxygen or sulfur atom; or an 8-, 9- or 10-membered fused aromatic or heteroaromatic ring system containing 0, 1, 2 or 3 nitrogen, oxygen or sulfur atoms, wherein there is no more than 2 oxygen or sulfur atoms;
wherein if Ar² is unsubstituted phenyl, then Ar¹ is not pyrazolyl;
wherein the aromatic rings Ar¹ and Ar² are substituted with 0, 1, 2 or 3 substituents selected from halogen, C₁₋₄alkyl, C₂₋₄alkenyl, C₂₋₄alkynyl, CN, NO₂, NR¹R², CH₂NR¹R², OR³, CH₂OR³, CO₂R⁴ and CF₃; but
if Ar¹ is phenyl and Ar² is quinolynyl, then Ar² is substituted with 0, 1, 2 or 3 substituents selected from C₁₋₄alkyl, C₂₋₄alkenyl, C₂₋₄alkynyl, CN, NO₂, NR¹R², CH₂NR¹R², OR³, CH₂OR³ and CO₂R⁴;
R¹, R², and R³ are independently C₁₋₄alkyl, aryl, heteroaryl, C(O)R⁵, C(O)NHR⁶, C(O)R⁷, SO₂R⁸; or R¹ and R² may together be (CH₂)ⱼG(CH₂)ₖ where G is oxygen, sulfur, NR^{9,} or a single bond;
j is 2, 3 or 4;
k is 0, 1 or 2;
R⁴, R⁵, R⁶, R⁷, R⁸, R⁹, and R¹⁰ are independently C₁₋₄alkyl, aryl, or heteroaryl; or an enantiomer thereof and pharmaceutically acceptable salts thereof;
with the provisos that:
(1) if D represents oxygen, E represents a single bond, and A represents: 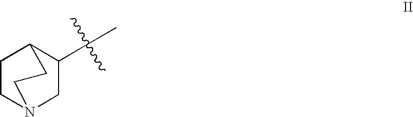
and either Ar¹ or Ar² represents a pyrazole ring, then all optional substituents on the pyrazole ring shall be hydrogen; and
(2) if Ar¹ represents a pyridine ring, Ar² represents an aryl ring, and A represents: 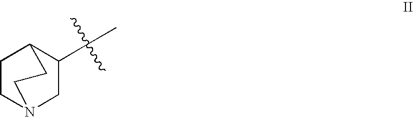
then all optional substituents on the pyridine ring shall be hydrogen.

5. The method according to claim 4, wherein said compound is selected from:
N-(1-azabicyclo[2.2.2]oct-3-yl)(3-phenylbenzamide);
N-(1-azabicyclo[2.2.2]oct-3-yl)(3-(2-thienyl)benzamide);
N-(1-azabicyclo[2.2.2]oct-3-yl)(3-(3-thienyl)benzamide);
N-(1-azabicyclo[2.2.2]oct-3-yl)(4-phenylthiophene-2-carboxamide);
N-(1-azabicyclo[2.2.2]oct-3-yl)(5-phenylthiophene-3-carboxamide);
N-(1-azabicyclo[2.2.2]oct-3-yl)(5-phenylthiophene-2-carboxamide);
N-(1-azabicyclo[2.2.2]oct-3-yl)(5-phenylfuran-2-carboxamide);
N-(1-azabicyclo[2.2.2]oct-3-yl)(5-(2-pyridyl)furan-2-carboxamide);
N-(1-azabicyclo[2.2.2]oct-3-yl)(5-(3-pyridyl)furan-2-carboxamide);
N-(1-azabicyclo[2.2.2]oct-3-yl)(5-(2-furyl)furan-2-carboxamide);
N-(1-azabicyclo[2.2.2]oct-3-yl)(5-(3-furyl)furan-2-carboxamide);
N-(1-azabicyclo[2.2.2]oct-3-yl)(5-(2-thienyl)furan-2-carboxamide);
N-(1-azabicyclo[2.2.2]oct-3-yl)(5-(3-thienyl)furan-2-carboxamide);
N-(1-azabicyclo[2.2.2]oct-3-yl)(5-(3-fluorophenyl)furan-2-carboxamide);
N-(1-azabicyclo[2.2.2]oct-3-yl)(3-(3-pyridyl)benzamide);
N-(1-azabicyclo[2.2.2]oct-3-yl)(3-(3-methoxyphenyl)benzamide);
N-(1-azabicyclo[2.2.2]oct-3-yl)(3-(2-methoxyphenyl)benzamide);
N-(1-azabicyclo[2.2.2]oct-3-yl)(3-(3-(N-acetylamino)phenyl)benzamide);
N-(1-azabicyclo[2.2.2]oct-3-yl)(3-(3-fluorophenyl)benzamide);
N-(1-azabicyclo[2.2.2]oct-3-yl)(3-(3-methylphenyl)benzamide);
N-(1-azabicyclo[2.2.2]oct-3-yl)(3-(3,5-dichlorophenyl)benzamide);
N-(1-azabicyclo[2.2.2]oct-3-yl)(3-(2-naphthyl)benzamide);
N-(1-azabicyclo[2.2.2]oct-3-yl)(3-(4-fluorophenyl)benzamide);
N-(1-azabicyclo[2.2.2]oct-3-yl)(5-(2-benzo[b]furanyl)furan-2-carboxamide);
N-(1-azabicyclo[2.2.2]oct-3-yl)(5-(4-pyridyl)furan-2-carboxamide);
N-(1-azabicyclo[2.2.2]oct-3-yl)(5-(3-methoxyphenyl)furan-2-carboxamide);
N-(1-azabicyclo[2.2.2]oct-3-yl)(5-(2-methoxyphenyl)furan-2-carboxamide);
N-(1-azabicyclo[2.2.2]oct-3-yl)(5-(4-fluorophenyl)furan-2-carboxamide);
N-(1-azabicyclo[2.2.2]oct-3-yl)(5-(2-naphthyl)furan-2-carboxamide);
N-(1-azabicyclo[2.2.2]oct-3-yl)(5-(3-methylphenyl)far an-2-carboxamide);
N-(1-azabicyclo[2.2.2]oct-3-yl)(5-(4-pyridyl)thiophene-2-carboxamide);
N-(1-azabicyclo[2.2.2]oct-3-yl)(5-(3-pyridyl)thiophene-2-carboxamide);
N-(1-azabicyclo[2.2.2]oct-3-yl)(5-(2-pyridyl)thiophene-2-carboxamide);
N-(1-azabicyclo[2.2.2]oct-3-yl)(4-(2-pyridyl)thiophene-2-carboxamide);
N-(1-azabicyclo[2.2.2]oct-3-yl)(4-(4-pyridyl)thiophene-2-carboxamide);
N-(1-azabicyclo[2.2.2]oct-3-yl)(4-(3-pyridyl)thiophene-2-carboxamide);
N-(1-azabicyclo[2.2.2]oct-3-yl)(5-(3-(N-acetylamino)phenyl)furan-2-carboxamide);
N-(1-azabicyclo[2.2.2]oct-3-yl)(5-(3-nitrophenyl)furan-2-carboxamide);
N-(1-azabicyclo[2.2.2]oct-3-yl)(5-(3-trifluoromethylphenyl)furan-2-carboxamide);
N-(1-azabicyclo[2.2.2]oct-3-yl)(5-(3-chlorophenyl)furan-2-carboxamide);
N-(1-azabicyclo[2.2.2]oct-3-yl)(5-(3-(N-acetylamino)phenyl)thiophene-2-carboxamide);
N-(1-azabicyclo[2.2.2]oct-3-yl)(5-(3-fluorophenyl)thiophene-2-carboxamide);
N-(1-azabicyclo[2.2.2]oct-3-yl)(5-(3-methoxyphenyl)thiophene-2-carboxamide);
N-(1-azabicyclo[2.2.2]oct-3-yl)(5-(3-ethoxyphenyl)thiophene-2-carboxamide);
N-(1-azabicyclo[2.2.2]oct-3-yl)(5-(3,5-dimethylisoxazol-4-yl)furan-2-carboxamide);
N-(1-azabicyclo[2.2.2]oct-3-yl)(5-(3,5-dimethylisoxazol-4-yl)thiophene-2-carboxamide);
N-(1-azabicyclo[2.2.2]oct-3-yl)(5-(3-aminophenyl)thiophene-2-carboxamide);
N-(1-azabicyclo[2.2.2]oct-3-yl)(5-(3-pyridyl)thiophene-3-carboxamide);
N-(1-azabicyclo[2.2.2]oct-3-yl)(5-(4-chlorophenyl)furan-2-carboxamide);
N-(1-azabicyclo[2.2.2]oct-3-yl)(5-(3-pyridyl)thiazole-3-carboxamide);
N-(1-azabicyclo[2.2.2]oct-3-yl)(5-(4-pyridyl)thiazole-3-carboxamide);
N-(1-azabicyclo[2.2.2]oct-3-yl)(5-(3-(N,N-dimethylamino)phenyl)thiophene-2-carboxamide);
N-(1-azabicyclo[2.2.2]oct-3-yl)(5-(8-quinolinyl)thiophene-2-carboxamide);
N-(1-azabicyclo[2.2.2]oct-3-yl)(5-(3-cyanophenyl)thiophene-2-carboxamide);
N-(1-azabicyclo[2.2.2]oct-3-yl)(5-(3-(N-methylamino)phenyl)thiophene-2-carboxamide);
N-(1-azabicyclo[2.2.2]oct-3-yl)(5-(3-hydroxyphenyl)thiophene-2-carboxamide);
N-(1-azabicyclo[2.2.2]oct-3-yl)(5-(3-pyridylamino)thiophene-2-carboxamide);
N-(1-azabicyclo[2.2.2]oct-3-yl)(5-(3-chlorophenyl)thiophene-2-carboxamide);
N-(1-aza-bicyclo[2.2.2]oct-3-yl)(5-(3-(4-morpholinyl)phenyl)thiophene-2-carboxamide);
N-(1-azabicyclo[2.2.2]oct-3-yl)(5-(3-(aminomethyl)phenyl)thiophene-2-carboxamide);
N-(1-azabicyclo[2.2.2]oct-3-yl)(5-phenoxythiophene-2-carboxamide);
N-(1-azabicyclo[2.2.2]oct-3-yl)(5-(3-aminophenyl)furan-2-carboxamide);
N-(1-azabicyclo[2.2.2]oct-3-yl)(5-(3-(N,N-dimethylamino)phenyl)furan-2-carboxamide);
N-(1-azabicyclo[2.2.2]oct-3-yl)(5-(3-formylphenyl)thiophene-2-carboxamide), or
N-(1-azabicyclo[2.2.2]oct-3-yl)(5-(3-(hydroxymethyl)phenyl)thiophene-2-carboxamide)
or an enantiomer thereof, or a pharmaceutically-acceptable salt thereof.

6. The method according to claim 1, wherein the α7 agonist is a compound having the structure: 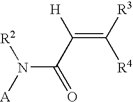
wherein:
A represents a moiety selected from: 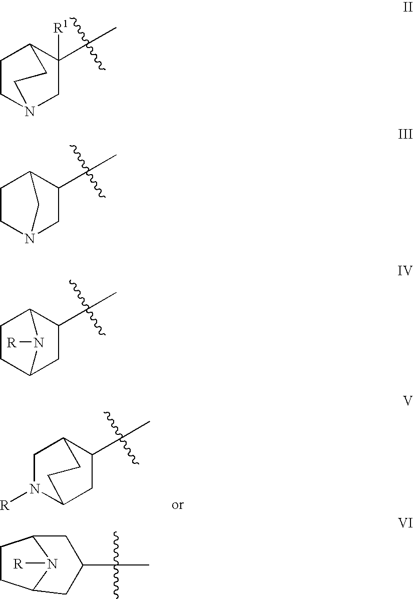
R represents hydrogen or methyl;
R¹ and R² are independently hydrogen, or C₁-C₄ alkyl;
R³ and R⁴ are independently hydrogen, C₁-C₄ alkyl or SAr, provided that at least one of R³ and R⁴ is SAr;
Ar represents a 5- or 6-membered aromatic or heteroaromatic ring containing zero to three nitrogen atoms, zero or one oxygen atom, and zero or one sulfur atom or an 8-, 9- or 10-membered fused aromatic or heteroaromatic ring system containing zero to four nitrogen atoms, zero to one oxygen atom, and zero to one sulfur atom which may optionally be substituted with one or more substituents selected from: hydrogen, halogen, C₁-C₄ alkyl, C₂-C₄ alkenyl, C₂-C₄ alkynyl, aryl, heteroaryl, —CO₂R⁵, —CN, —NO₂, —NR⁶R⁷, —CF₃, —OR⁸;
R⁵, R⁶, R⁷, and R⁸ are independently hydrogen, C₁-C₄ alkyl, aryl, heteroaryl, —C(O)R⁹, —C(O)NHR¹⁰, —C(O)R¹¹, —SO₂R¹²; or,
R⁶ and R⁷ may together be (CH₂)ⱼQ(CH₂)ₖ where Q is O, S, NR¹³, or, a bond;
j is 2 to 7;
k is 0 to 2;
R⁹, R¹⁰, R¹¹, R¹², and R¹³, are independently C₁-C₄ alkyl, aryl, or heteroaryl;
or an enantiomer thereof, and the pharmaceutically acceptable salts thereof.

7. The method according to claim 6, wherein said compound is selected from:
N-(1-aza-bicyclo[2.2.2]oct-3-yl)[Z-3-(phenylthio)propenamide]hydrochloride;
N-(1-aza-bicyclo[2.2.2]oct-3-yl)[Z-3-(4-methylphenylthio)propenamide];
N-(1-aza-bicyclo[2.2.2]oct-3-yl)[E-3-(4-methylphenylthio)propenamide];
N-(1-aza-bicyclo[2.2.2]oct-3-yl)[Z-3-(3-methylphenylthio)propenamide];
N-(1-aza-bicyclo[2.2.2]oct-3-yl)[E-3-(3-methylphenylthio)propenamide];
N-(1-aza-bicyclo[2.2.2]oct-3-yl)[Z-3-(2-methylphenylthio)propenamide];
N-(1-aza-bicyclo[2.2.2]oct-3-yl)[E-3-(2-methylphenylthio)propenamide];
N-(1-aza-bicyclo[2.2.2]oct-3-yl)[Z-3-(4-methoxyphenylthio)propenamide];
N-(1-aza-bicyclo[2.2.2]oct-3-yl)[E-3-(4-methoxyphenylthio)propenamide];
N-(1-aza-bicyclo[2.2.2]oct-3-yl)[Z-3-(3-methoxyphenylthio)propenamide];
N-(1-aza-bicyclo[2.2.2]oct-3-yl)[E-3-(3-methoxyphenylthio)propenamide];
N-(1-aza-bicyclo[2.2.2]oct-3-yl)[Z-3-(2-methoxyphenylthio)propenamide];
N-(1-aza-bicyclo[2.2.2]oct-3-yl)[E-3-(2-methoxyphenylthio)propenamide];
N-(1-aza-bicyclo[2.2.2]oct-3-yl)[Z-3-(2-pyridylthio)propenamide];
N-(1-aza-bicyclo[2.2.2]oct-3-yl)[E-3-(2-pyridylthio)propenamide];
N-(1-aza-bicyclo[2.2.2]oct-3-yl)[Z-3-(4-pyridylthio)propenamide];
N-(1-aza-bicyclo[2.2.2]oct-3-yl)[E-3-(4-pyridylthio)propenamide];
N-(1-aza-bicyclo[2.2.2]oct-3-yl)[Z-3-(2-pyrimidinylthio)propenamide];
N-(1-aza-bicyclo[2.2.2]oct-3-yl)[E-3-(2-pyrimidinylthio)propenamide];
N-(1-aza-bicyclo[2.2.2]oct-3-yl)[Z-3-(2-methyl-3-furanylthio)propenamide];
N-(1-aza-bicyclo[2.2.2]oct-3-yl)[E-3-(2-methyl-3-furanylthio)propenamide];
N-(1-aza-bicyclo[2.2.2]oct-3-yl)[E-3-(2-imidazolylthio)propenamide];
N-(1-aza-bicyclo[2.2.2]oct-3-yl)[Z-3-(phenylthio)-3-(methyl)propenamide];
N-(1-aza-bicyclo[2.2.2]oct-3-yl)[Z-3-(2-benzothiazolylthio)propenamide];
N-(1-aza-bicyclo[2.2.2]oct-3-yl)[E-3-(2-benzothiazolylthio)propenamide];
N-(1-aza-bicyclo[2.2.2]oct-3-yl)[Z-3-(1-methyl-2-imidazolylthio)propenamide];
N-(1-aza-bicyclo[2.2.2]oct-3-yl)[E-3-(1-methyl-2-imidazolylthio)propenamide];
N-(1-aza-bicyclo[2.2.2]oct-3-yl)[Z-3-(5-methyl-1,3,4-thiadiazol-2-ylthio)propenamide];
N-(1-aza-bicyclo[2.2.2]oct-3-yl)[E-3-(5-methyl-1,3,4-thiadiazol-2-ylthio)propenamide];
N-(1-aza-bicyclo[2.2.2]oct-3-yl)[Z-3-(4-chlorophenylthio)propenamide];
N-(1-aza-bicyclo[2.2.2]oct-3-yl)[Z-3-(2-thiazolylthio)propenamide];
N-(1-aza-bicyclo[2.2.2]oct-3-yl)[Z-3-(2-thienylthio)propenamide];
N-(1-aza-bicyclo[2.2.2]oct-3-yl)[E-3-(2-thienylthio)propenamide];
N-(1-aza-bicyclo[2.2.2]oct-3-yl)[Z-3-(2-benzoxazolylthio)propenamide];
N-(1-aza-bicyclo[2.2.2]oct-3-yl)[E-3-(2-benzoxazolylthio)propenamide];
N-(1-aza-bicyclo[2.2.2]oct-3-yl)[Z-3-(4-trifluoromethyl-2-pyrimidinylthio)propenamide];
N-(1-aza-bicyclo[2.2.2]oct-3-yl)[Z-3-(4-fluorophenylthio)propenamide];
N-(1-aza-bicyclo[2.2.2]oct-3-yl)[E-3-(4-fluorophenylthio)propenamide];
N-(1-aza-bicyclo[2.2.2]oct-3-yl)[Z-3-(2-thiazolo[4,5-b;pyridylthio)propenamide];
(R)—N-(1-aza-bicyclo[2.2.2]oct-3-yl)[E-3-(2-thiazolo[4,5-b]pyridylthio)propenamide];
N-(1-aza-bicyclo[2.2.2]oct-3-yl)[Z-3-(3-fluorophenylthio)propenamide], or
N-(1-aza-bicyclo[2.2.2]oct-3-yl)[E-3-(3-fluorophenylthio)propenamide];
or an enantiomer thereof, or a pharmaceutically-acceptable salt thereof

8. The method according to claim 1, wherein the α7 agonist is a compound having the structure: 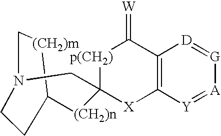
wherein n is 0 or 1;
m is 0 or 1;
p is 0 or 1;
X is oxygen or sulfur;
Y is CH, N or NO;
W is oxygen, H₂ or F₂;
A is N or C(R²);
G is N or C(R³);
D is N or C(R⁴);
with the proviso that no more than one of A, G, and D is nitrogen but at least one of Y, A, G, and D is nitrogen or NO; R¹is hydrogen or C₁₋₄alkyl;
R², R³, and R⁴ are independently hydrogen, halogen, C₁₋₄alkyl, C₂₋₄alkenyl, C₂₋₄alkynyl, aryl, heteroaryl, OH, OC₁₋₄alkyl, CO₂R¹, —CN, —NO₂, —NR⁵R⁶, —CF₃, —OSO₂CF₃, or R² and R³, or R³ and R⁴, respectively, may together form another six membered aromatic or heteroaromatic ring sharing A and G, or G and D, respectively, containing 0, 1 or 2 nitrogen atoms, and substituted with one to two substituents independently selected from hydrogen, halogen, C₁₋₄alkyl, C₂₋₄alkenyl, C₂₋₄alkynyl, aryl, heteroaryl, OH, OC₁₋₄alkyl, CO₂R¹, —CN, —NO₂, —NR⁵R⁶, —CF₃, —OSO₂CF₃;
R⁵ and R⁶ are independently hydrogen, C₁₋₄alkyl, C(O)R⁷, C(O)NHR⁸, C(O)OR⁹, SO₂R¹⁰ or may together be (CH₂)ⱼQ(CH₂)ₖ where Q is O, S, NR¹¹, or a bond;
j is 2 to 7;
k is 0 to 2;
R⁷, R⁸, R⁹, R¹⁰, and R¹¹ are independently C₁₋₄alkyl, aryl, or heteroaryl, or an enantiomer thereof, and the pharmaceutically acceptable salts thereof.

9. The method according to claim 8, wherein said compound is selected from:
spiro[1-azabicyclo[2.2.2]octane-3,2′-(3′H)-furo[2,3-b]pyridine];
5′-bromospiro[1-azabicyclo[2.2.2]octane-3,2′-(3′H)-furo[2,3-b]pyridine];
5′-phenylspiro[1-azabicyclo[2.2.2]octane-3,2′-(3′H)-furo[2,3-b]pyridine];
5′-nitrospiro[1-azabicyclo[2.2.2]octane-3,2′-(3′H)- furo[2,3-b]pyridine];
1′-chlorospiro[1-azabicyclo[2.2.2]octane-3,2′-(3′H)-furo[2,3-b]isoquinoline];
5′-(phenylcarboxamido)spiro[1-azabicyclo[2.2.2]octane-3,2′-(3′H)-furo[2,3-b]pyridine];
5′-(phenylaminocarbonylamino)spiro[1-azabicyclo[2.2.2]octane-3,2′-(3′H)-furo[2,3-b]pyridine];
5′-(phenylsulfonylamido)spiro[1-azabicyclo[2.2.2]octane-3,2′-(3′H)-furo[2,3-b]pyridine];
5′-aminospiro[1-azabicyclo[2.2.2]octane-3,2′-(3′H)-furo[2,3-b]pyridine];
5′-N-methylaminospiro[1-azabicyclo[2.2.2]octane-3,2′-(3′H)-furo[2,3-b]pyridine];
5′-N,N-dimethylaminospiro[1-azabicyclo[2.2.2]octane-3,2′-(3′H)-furo[2,3-b]pyridine];
5N,N-diethylaminospiro[1-azabicyclo[2.2.2]octane-3,2′-(3′H)-furo[2,3-b]pyridine];
5′-N-ethylaminospiro[1-azabicyclo[2.2.2]octane-3,2′-(3′H)-furo[2,3-b]pyridine];
5′-N-benzylaminospiro[1-azabicyclo[2.2.2]octane-3,2′-(3′H)-furo[2,3-b]pyridine];
5′-N-formamidospiro[1-azabicyclo[2.2.2]octane-3,2′-(3′H)-furo[2,3-b]pyridine];
5′-N-acetamidospiro[1-azabicyclo[2.2.2]octane-3,2′-(3′H)-furo[2,3-b]pyridine];
spiro[1-azabicyclo[2.2.2]octane-3,2′-(3′H)-furo[2,3-b]isoquinoline];
spiro[1-azabicyclo[2.2.2]octane-3,2′-(3′H)-furo[2,3-b]quinoline];
5′-ethenylspiro[1-azabicyclo[2.2.2]octane-3,2′-(3′H)-furo[2,3-b]pyridine];
5′-(E)-(phenylethenyl)spiro[1-azabicyclo[2.2.2]octane-3,2′-(3′H)-furo[2,3-b]pyridine];
5′-(4-morpholino)spiro[1-azabicyclo[2.2.2]octane-3,2′-(3′H)-furo[2,3-b]pyridine];
5′-(1-azetidinyl)spiro[1-azabicyclo[2.2.2]octane-3,2′-(3′H)-furo[2,3-b]pyridine];
5′-(E)-(2-(4-pyridyl)ethenyl)spiro[1-azabicyclo[2.2.2]octane-3,2′-(3′H)-furo[2,3-b]pyridine];
5′-(E)-(2-(2-pyridyl)ethenyl)spiro[1-azabicyclo[2.2.2]octane-3,2′-(3′H)-furo[2,3-b]pyridine];
5′-(2-trimethylsilylethynyl)spiro[1-azabicyclo[2.2.2]octane-3,2′-(3′H,)-furo[2,3-b]pyridine];
5′-ethynylspiro[1-azabicyclo[2.2.2]octane-3,2′-(3′H)-furo[2,3-b]pyridine];
5′-(2-furyl)spiro[1-azabicyclo[2.2.2]octane-3,2′-(3′H)-furo[2,3-b]pyridine];
5′-(3-pyridyl)spiro[1-azabicyclo [2.2.2]octane-3,2′-(3′H)-furo[2,3-b]pyridine];
5′-methylspiro[1-azabicyclo[2.2.2]octane-3,2′-(3′H)-furo[2,3-b]pyridine];
spiro[1-azabicyclo[2.2.2]octane-3,2′-(3′H)-furo[2,3-b]pyridine-5′carbonitrile];
spiro[1-azabicyclo[2.2.2]octane-3,2′-(3′H)-furo[2,3-b]pyridine-5′carboxamide];
5′-N′-(3-chlorophenyl)aminocarbonylminospiro[1-azabicyclo[2.2.2]octane-3, 2′-(3′H)-furo[2,3-b]pyridine];
5′-N′-(2-nitrophenyl)aminocarbonylaminospiro[1-azabicyclo[2.2.2]octane-3, 2′-(3 ′H)-furo[2,3-b]pyridine];
4′-chlorospiro[1-azabicyclo[2.2.2]octane-3,2′-(3′H)-furo[2,3-b]pyridine];
4′-methoxyspiro[1-azabicyclo[2.2.2]octane-3,2′-(3′H)-furo [2,3-b]pyridine];
4′-phenylthiospiro[1-azabicyclo[2.2.2]octane-3,2′-(3′H)-furo[2,3-b]pyridine];
4′-(N-2-aminoethyl)aminospiro[1-azabicyclo[2.2.2]octane-3,2′-(3′H)-furo[2,3-b]pyridine]
4′-phenylaminospiro[1-azabicyclo[2.2.2]octane-3,2′-(3′H)-furo[2,3-b]pyridine];
4′-methylaminospiro[1-azabicyclo[2.2.2]octane-3,2′-(3′H)-furo[2,3-b]pyridine];
4′-(4-N-methylpiperazin-1-yl)spiro[1-azabicyclo[2.2.2]octane-3, 2′-(3′H)-furo[2,3-b]pyridine];
4′-chloro-spiro[1-azabicyclo[2.2.2]octane-3,2′-(3′H)-furo[3,2-c]pyridine];
spiro[1-azabicyclo[2.2.2]octane-3,2′-(3′H)-furo[3,2-c]pyridine];
spiro[1-azabicyclo[2.2.2]octane-3,2′(3′H)-furo [2,3-b]pyridine-7′-oxide];
spiro[1-azabicyclo[2.2.2]octane-3,2′(3′H)-furo[2,3-b]pyridine-6′-carbonitrile];
6′-chlorospiro[1-azabicyclo[2.2.2]octane-3,2′(3′H)-fire[2,3-b]pyridine], or
6′-fluorospiro[1-azabicyclo[2.2.2]octane-3,2′(3′H)-furo[2,3-b]pyridine];
or an enantiomer, or a pharmaceutically-acceptable salt thereof.

10. The use of an α7 antagonist for the manufacture of a medicament for the treatment or prophylaxis of fibromyalgia syndrome and fibromyalgia-related symptoms comprising an α7 agonist.

11. The use according to claim 10, wherein the α7 agonist is a compound that has a Kᵢ value of less than 1000 nM in the ¹²⁵I-α-Bungarotoxin binding to rat hippocampal membrane assay.

12. The use of an α7 antagonist for the manufacture of a medicament comprising an α7 agonist compound having a structure according to any one of claims 3, 4, 5, 6, 7, 8 or 9.
